(19) United States
(12) Patent Application Publication
(10) Pub. No.: US 2008/0243103 A1
(43) Pub. Date: Oct. 2, 2008

(54) Medical Device for Delivering a Bioactive and Method of Use Thereof

(75) Inventors: Daniel K. Whetham, Bloomington, IN (US); Jessica Watts Miller, St. Louis, MO (US); Andrew P. Isch, Lafayette, IN (US); Brandt M. Davis, Lafayette, IN (US); Steven Pendleton, Spencer, IN (US)

Correspondence Address: BRINKS HOFER GILSON & LIONE/CHICAGO/COOK, PO BOX 10395, CHICAGO, IL, 60610, US

(73) Assignees: Cook Urological Inc., Spencer, IN (US); MED Institute, Inc., West Lafayette, IN (US)

(21) Appl. No.: 12/058,206

(22) Filed: Mar. 28, 2008

Related U.S. Application Data
(60) Provisional application No. 60/920,535, filed on Mar. 28, 2007.

(51) Int. Cl. A61M 31/00; A61M 29/02

(52) U.S. Cl. 604/515; 604/101.02; 604/103.02

(57) ABSTRACT

A device and method for delivering a bioactive to the uterine wall. In one embodiment, the device comprises a balloon having the bioactive present within the material of the balloon or on an outside surface of the balloon. Another embodiment provides a method comprising inserting the balloon into the uterus, inflating the balloon so that the outside surface of the balloon exerts pressure on uterine wall and delivering the bioactive to the uterine wall.

### RELATED APPLICATIONS

This application claims the benefit of the filing date under 35 U.S.C. §119(e) of Provisional U.S. Patent Application Ser. No. 60/920,535, filed Mar. 28, 2007, the contents of which are hereby incorporated by reference.

### TECHNICAL FIELD

This invention relates generally to medical devices for delivering a bioactive to a body cavity of a human or veterinary patient and methods of using such devices. In one embodiment, a medical device delivers a bioactive to the uterine wall to control hemorrhaging.

### BACKGROUND OF THE INVENTION

Postpartum bleeding is bleeding that occurs immediately after the placenta is delivered. With an incidence of one in twenty deliveries, it is one of the leading causes of maternal mortality. In cases of severe bleeding where hemorrhaging may cause a threat to life, hysterectomy remains the conventional method of treatment. However, more conservative approaches are becoming increasingly proposed as an alternative to hysterectomy.

One such approach involves the application of pressure to the uterine wall to prevent bleeding. Manual compression of the utorine wall may be sufficient to staunch the flow of blood, at least temporarily. If this procedure is not sufficient, alternative methods of applying compression to the uterine wall may be called for.

Uterine packing is one such method that has been used successfully to manage postpartum bleeding. However, this technique often fails due to the difficulty in packing the uterus so that an even pressure in applied to the uterine wall. Furthermore, removing the packing material can sometimes require a separate surgical procedure. Because of these difficulties, uterine packing is not considered an ideal treatment method.

As an alternative to uterine packing, an inflatable balloon may be inserted into the uterus and inflated sufficiently to apply pressure to the uterine wall. The Sengstaken-Blakemore balloon, a naso-gastric balloon for tamponade of esophageal varicoceles, has been successfully used for the treatment of postpartum bleeding. However, this balloon is not specifically intended for application to the uterus and does not necessarily conform to the shape of the uterus. In addition, this balloon contains latex and may therefore cause allergic reactions in some patients.

The Bakri Postpartum Balloon Catheter (Cook Women's Health, Spencer, Indiana 47460) is specifically designed for the treatment of postpartum bleeding. This device is manufactured from silicone and contains no latex. It has a ductile shape which allows the inflated balloon to conform to the shape of the uterus. The Bakri balloon allows for hemostatic cushion application and limits clot formation. A large diameter lumen built into the shaft and a multi-ported, non-abrasive tip allows for constant drainage, so that an ongoing uterine hemorrhage does not go undetected. Finally, once deflated the Bakri balloon may be easily removed without the need for an additional surgical procedure.

Clinically, 75-80% of postpartum hemorrhages are due to uterine atony. Normally, contraction of the uterine arteries compresses the surrounding vessels and reduces blood flow. However, a loss of tone in the uterine musculature (uterine atony) can result in an absence of the normal contraction and a decreased likelihood of coagulation. The lack of normal uterine muscle contraction can cause an acute hemorrhage.

In cases of uterine atony, uterotonic agents, such as prostaglandins, have been administered in an attempt to stimulate uterine contraction. Various modes of administration have been proposed, including intramuscular injection, intravenous injection, and oral, rectal, or vaginal suppository. However, the proper delivery of agents such as these is a clinical determination of a licensed physician.

U.S. Pat. No. 6,676,680, issued on Jan. 13, 2004 to Packer, discloses a tamponade device for controlling postpartum hemorrhaging. This device includes a balloon that may be coated or impregnated with a hemostatic material, such as oxidized cellulose or hemotene, in order to further control bleeding.

U.S. Pat. No. 5,749,845, issued on May 12, 1998 to Hildebrand and Shapland, discloses a method for delivering an agent to the bladder or the uterus. The method utilizes a catheter having an inflatable portion. The inflatable portion is inserted into the organ and is inflated so that it expands the organ. The agent is then delivered from the inflatable portion into the wall of the organ.

Apart from the treatment of postpartum bleeding, a number of other medical procedures performed on the uterus may call for a device capable of reducing or preventing bleeding. One such device is a balloon uterine stent, such as that available from Cook Women's Health, Spencer, Indiana 47460. Such devices may be placed in the uterus following an intrauterine surgical procedure to reduce uterine bleeding by applying pressure to the uterine wall.

### SUMMARY OF THE INVENTION

The foregoing problems are solved and a technical advance is achieved in an improvement to a medical device that is implantable either partly or completely into a human or veterinary patient. One aspect of the present invention provides a delivery system for a bioactive. In one embodiment, the delivery system includes a catheter having a first lumen and two inflatable balloons, one positioned within the other. The inner balloon is in fluid communication with the first lumen. A composition including a bioactive is positioned within an intermediate space between the balloons. In one embodiment, the bioactive is an uterotonic agent In one embodiment, the outer inflatable balloon is at least partially permeable to the bioactive and the inner inflatable balloon is impermeable to the bioactive.

In another embodiment, the bioactive is an agent for the treatment of abnormal uterine bleeding, post-hysteroscopic tamponade, post-partum hemorrhage, Ascherman's syndrome or endometrial ablation. In yet another embodiment, the bioactive is a prostaglandin or a prostaglandin analogue. In another embodiment, the bioactive is 15-methyl prostaglandin F_{2α}, a synthetic prostaglandin E₁ (PGE₁) analogue, ergometrine maleate or oxytocin or a mixture thereof.

In another embodiment, at least one of the balloons includes a polymer. The polymer can be polyurethane, silicone, natural rubber, synthetic rubber or latex.

In another embodiment, the delivery system includes a catheter having a lumen, an inflatable balloon having its inside surface in fluid communication with the lumen and an uterotonic agent positioned within the material forming the inflatable balloon or on the outside surface of the inflatable balloon.

In yet another embodiment, the delivery system also includes a gauze or resorbable mesh attached to the outside surface of the balloon. The bioactive is contained within the gauze or resorbable mesh.

Another aspect of the present invention provides a method of treating postpartum hemorrhage. The method includes inserting an inflatable balloon into the uterus. The balloon is attached to a catheter having a lumen in fluid communication with the balloon. An uterotonic agent is present within material forming the balloon or on the outside surface of the balloon. The balloon is inflated to contact and apply pressure to the uterine wall. Contact is maintained for a time sufficient to deliver a therapeutically effective amount of the uterotonic agent to the uterine wall. In one embodiment, the bioactive is a prostaglandin or a prostaglandin analogue. In another embodiment, the bioactive is 15-methyl prostaglandin F_{2α}, a synthetic prostaglandin E₁ (PGE₁) analogue, ergometrine maleate or oxytocin or a mixture thereof.

In yet another embodiment, contact time with the uterine wall is less than 60 minutes. In another embodiment, contact time with the uterine wall is less than 30 minutes.

### BRIEF DESCRIPTION OF THE DRAWINGS

FIG. 1A depicts a partial cross-section view of one embodiment of the device of the present invention;

FIG. 1B depicts a partial cross-section view of another embodiment of the device of the present invention;

FIG. 2 depicts a partial cross-section view of yet another embodiment of the device of the present invention;

FIG. 3 depicts a partial cross-section view of another embodiment of the device of the present invention;

FIG. 4 depicts a cross-section view of another embodiment of the device of the present invention.

### DETAILED DESCRIPTION

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

The terms “about” or “substantially” used with reference to a quantity includes variations in the recited quantity that are equivalent to the quantity recited, such as an amount that is insubstantially different from a recited quantity for an intended purpose or function.

As used herein, the term “implantable” refers to an ability of a medical device to be positioned, partially or wholly, at a location within a body of a human or veterinary patient for any suitable period of time, such as within a body cavity. For example, the medical device may be implanted within the uterus, esophagus, trachea, colon, biliary tract, urinary tract, or vascular system of a patient. Furthermore, the terms “implantation” and “implanted” refer to the positioning of a medical device, partially or wholly, at a location within a body, such as within a body vessel. Implantable medical devices can be configured for transient placement within a body vessel during a medical intervention (e.g., minutes to hours), or to remain in a body vessel for a prolonged period of time after an implantation procedure (e.g., weeks or months or years). Implantable medical devices can include devices configured for bioabsorption within a body during a prolonged period of time.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response. Such a response is distinguished from a mild, transient inflammation which typically accompanies surgery or implantation of foreign objects into a living organism.

As used herein, the term “bioactive” refers to any agent that produces an intended therapeutic effect on the body to treat or prevent conditions or diseases.

As used herein, a “mixture” refers to a combination of two or more ingredients in which each ingredient retains its own chemical identity and properties.

The phrase “controlled release” refers to the release of a material, such as a bioactive, from a device at a predetermined rate. The predetermined rate may be determined by, for example, the presence of a carrier material mixed with the bioactive and/or the presence of a barrier layer between the bioactive and the surface of the device. In general, the rate of controlled release will be such that the material is released over a longer period than would be the case if the carrier material and/or barrier layer were not present. A controlled release may be constant or vary with time.

A controlled release may be characterized by an elution profile, which shows the measured rate that the material is removed from the device in a given solvent environment as a function of time. For example, a controlled release elution profile may include an initial burst release associated with the deployment of the medical device, followed by a more gradual subsequent release. A controlled release may be a gradient release in which the concentration of the material released varies over time or a steady state release in which the material is released in equal amounts over a certain period of time (with or without an initial burst release).

As used herein, a “barrier layer” is any layer that is placed over at least a portion of a bioactive present in or on a device. In general, the Ibioactive will not be present in the barrier layer. Any mixing of a bioactive with the barrier layer is unintentional and merely incidental. The barrier layer may or may not be the outer-most layer present on the device. For example, a bioactive may be coated onto a surface of a device, a first barrier layer placed over the bioactive and further barrier layers and layers containing the same or a different bioactive placed on the first barrier layer. The barrier layer may control the release of the bioactive from the device upon implantation.

As used herein, a “carrier material” refers to a material that forms a mixture with bioactive on or in a device. The carrier material may control the release of the bioactive from the device.

The term “treatment” or “treating” as used herein describes the management and care of a human or veterinary patient for the purpose of combating or preventing a disease, condition, or disorder and includes procedures such as angioplasty and the administration of a bioactive to alleviate the symptoms or complications, or eliminate the disease, condition, or disorder.

A “therapeutically-effective amount” as used herein is the minimal amount of a bioactive (e.g. a prostaglandin) which is necessary to impart therapeutic benefit to a human or veterinary patient. For example, a “therapeutically effective amount” to a human or veterinary patient is such an amount which induces, ameliorates or otherwise causes an improvement in the pathological symptoms, disease progression or physiological conditions associated with or resistance to succumbing to a disorder, for example postpartum hemorrhaging.

### Implantable Medical Devices

One aspect of the present invention provides implantable medical devices for delivering a bioactive to the wall of a body cavity. Preferably, the bioactive is delivered to the uterine wall. However, such devices may be applied to the delivery of a bioactive to the wall of another organ, for example, the bladder or the esophagus. In one embodiment, such devices include a balloon which, when inflated, is adapted to contact and apply a pressure to a substantial portion of the wall of the uterus. The bioactive is incorporated into or onto the balloon portion of the device and is delivered from the inflated balloon to the cavity wall.

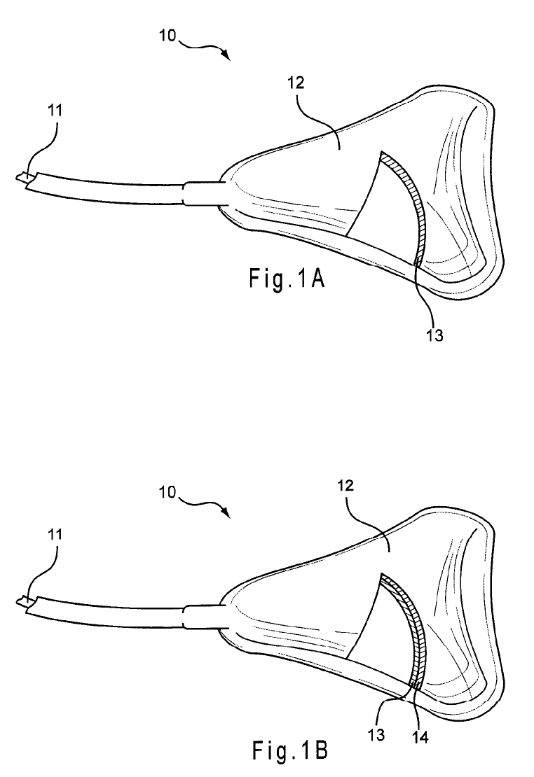

FIGS. 1(a) and 1(b) depict a partial cross section of one embodiment. Referring to FIG. 1(a), device 10 comprises an inflatable balloon portion 12 and an elongated catheter having an inflation lumen 11 in fluid communication with the inside of the balloon. Inflation lumen 11 allows for the inflation of balloon portion 12 by delivering a fluid to the inside of balloon portion 12 via inflation lumen 11. In one embodiment, the wall 13 of inflatable balloon 12 is impregnated with a bioactive. Alternatively, as is depicted in FIG. 1(b), the bioactive is present on the outside surface of balloon portion 12 in coating 14. Coating 14 may include a carrier material. For example, the bioactive may be present within a polymer coating or impregnated within a gauze or resorbable material.

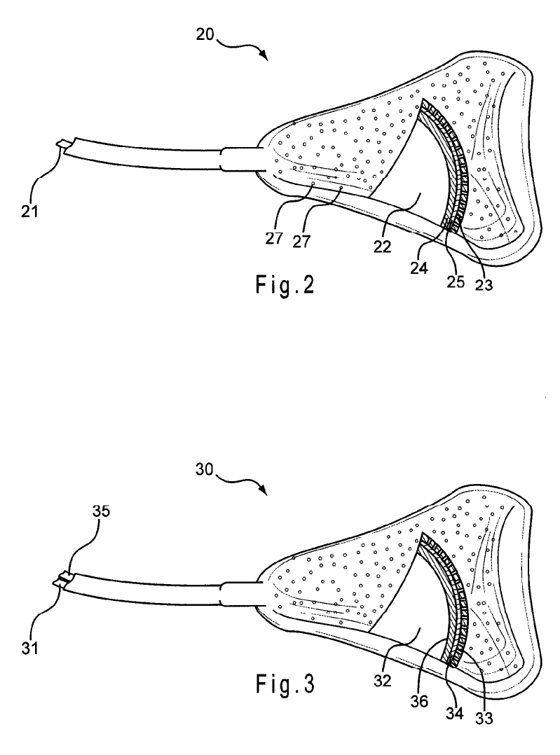

FIG. 2 depicts a partial cross section of another illustrative embodiment of the present invention. Device 20 comprises outer inflatable balloon 23 and inner inflatable balloon 24 positioned within outer inflatable balloon 23. An elongated catheter having an inflation lumen 21 is in fluid communication with the lumen 22 of inner inflatable balloon 23. Outer inflatable balloon 23 covers the outside surface of inner balloon 24 forming closed intermediate space 25 between the outer surface of balloon 24 and the inner surface of balloon 23. A bioactive is contained within intermediate space 25. In one embodiment, the bioactive is dissolved in a liquid or gel. In such an embodiment, balloon 24 is preferably impermeable to the liquid or gel bioactive formulation whereas balloon 23 is a least semi-permeable to this formulation. For example, balloon 23 can contain pores 27. Inflation of balloon 24 forces the bioactive through the wall onto the outer surface of balloon 23 and into contact with the uterine wall.

FIG. 3 depicts a partial cross section of yet another illustrative embodiment of the invention. This embodiment is similar to the embodiment disclosed in FIG. 2 and includes an elongated catheter having an inflation lumen 31 in fluid communication with the lumen 32 of inner inflatable balloon 36 and a bioactive delivery inlet lumen 35 in fluid communication with intermediate space 34 between outer balloon 33 and inner balloon 36. Bioactive delivery inlet lumen 35 allows for the delivery of a bioactive to intermediate space 34 when the device is implanted.

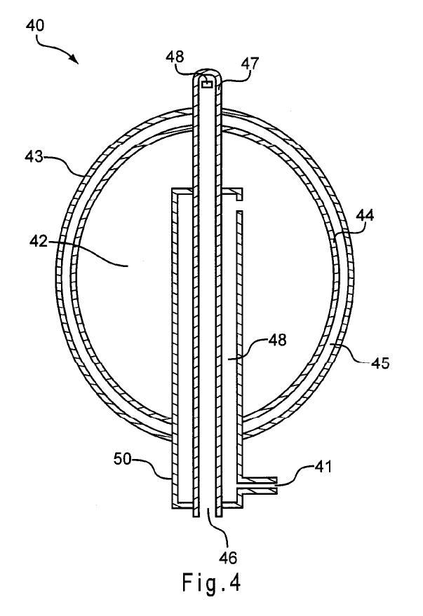

FIG. 4 depicts a cross-section of another illustrative embodiment of the invention depicts 40 comprises inflatable balloon 44 and an elongated catheter 50 having an inflation lumen 48 in communication with the interior of balloon 44 and with inflation port 41. Balloon 43 covers the outside surface of balloon 44 forming an intermediate space 45 between the outer surface of balloon 44 and the inner surface of balloon 43. Hollow drain tube 46 is coaxially positioned within balloons 44 and 43 and extends distally of these balloons to drainage port(s) 48 at distal end 47 of device 40. Balloons 43 and 44 are attached to drain tube 46 and elongated catheter 50 to form seals where the tube or catheter pierces the balloons. Drain tube 46 allows for constant drainage of any blood present the uterus, so that any ongoing uterine hemorrhage does not go undetected. Of course, other embodiments of the invention, as described above, including those described in FIGS. 1-3, can also encompass a drain tube, such as drain tube 46.

The balloon portions of the present devices are preferably manufactured from a silicone. However, other biocompatible materials can also be used. Such materials include, but are not limited to, biocompatible polymers such as polyethylenes, polyurethanes, nylons, polyesters, latex, natural rubber, synthetic rubber, elastomers and mixtures or copolymers of these materials.

In one embodiment, the balloon portion is adapted for the delivery of a bioactive for the treatment of postpartum hemorrhaging. A balloon used for the treatment of this condition is manufactured to have a volume such that, upon Inflation, the outside surface of the balloon exerts pressure to a substantial portion of the wall of the dilated uterus. Typically, the inflated volume of the balloon is in the range of 400 to 1000 cc, preferably between 400 and 700 cc, more preferably between 400 and 600 cc.

Another embodiment provides device for the treatment of conditions such an abnormal uterine bleeding. In such conditions, the uterus is normally not as voluminous as it is postpartum. Consequently, such devices include a balloon having a smaller inflated volume than is the case with devices used in the treatment of postpartum hemorrhage. Typically, the inflated volume of such a balloon is between 3 to 7 cc, preferably between 3 and 5 cc.

Another aspect provides a balloon having an absorbent present on the balloon surface. The absorbent contacts the wall of a body cavity upon inflation of the balloon within a body cavity. Such a balloon can be used to apply pressure on the cavity wall and/or absorb fluids present on the wall so that any bleeding is reduced or eliminated without the need of a bioactive. In various embodiments, the absorbent includes mesh cotton cellulose or derivative of cellulose, cotton, cotton derivatives, alginates, dextran or rayon. In another embodiment, the absorbent includes a powder form of a suitable extracellular matrix material (“ECM material”.) Such materials include, for instance, submucosa (including, for example, small intestinal submucosa (“SIS”), stomach submucosa, urinary bladder submucosa, or uterine submucosa), renal capsule membrane, dura matter, pericardium, serosa, and peritoneum or basement membrane materials, including liver basement membrane

### Bioactives

A vast range of drugs, medicaments and materials may be employed as a bioactive with the devices of the present invention. Of course, devices having two or more bioactives, such as two or more bioactives from those mentioned below, present in or on the balloon portion are encommpassed. It is intended that the term bioactive includes any material that is molecularly interactive with the fluids, cells, proteins or tissues of an animal including humans to augment the diagnosis, treatment or prevention of any physiologic or pathologic condition. It is further intended that this term includes therapeutic and diagnostic agents such as, for example, drugs, vaccines, hormones, steroids, proteins, previously described agents, complexing agents, salts, chemical compounds, polymers, and the like.

In one embodiment, the bioactive is an agent that induces contraction of the uterus and/or controls bleeding from the uterine wall. Such bioactives include uterotonic agents such as prostaglandins. In preferred embodiments, the bioactive is HEMABATE® (Carboprost tromethamine or 15-methyl prostaglandin F_{2α}), CYTOTEC® (misoprostol, a synthetic prostaglandin E₁ (PGE₁) analogue), SYNTOCINON® (synthetic oxytocin), CERVIDIL®, PROSTIN E2® (dinoprostone) or SYNTOMETRINE® (a combination of ergometrine and oxytocin). In another embodiment, the bioactive is aluminum sulfate anhydrous. In yet another embodiment, the bioactive is a flocculent or coagulant such as the natural chemical Chitosan.

Of course, bioactives having application in the treatment of other conditions can also be successfully delivered by the present devices. For example, an anti-cancer chemotherapeutic agent can be delivered. Such anticancer agents include, but are not limited to, tamoxifen citrate, TAXOL® (Paclitaxel) or derivatives thereof PROSCAR® (Finasteride), HYTRIN® (Terazosin), EULEXIN® (flutamide), docetaxel and its derivatives, fluoro-pyrimidines including 5-fluoroacil and its derivatives, hydroxyurea, mercaptopurine, cisplatin, anthracyclines including daunorubicin and doxorubicin and their derivatives, podophylotoxins including etoposide, and mitoxantrone and its derivatives, a folic acid antagonist other than methotrexate and its derivatives, a camptothecin, and a platinum complex.

In another embodiment, the bioactive is an antimicrobial drug such as a penicillin, cephalosporin, carbepenem, beta-lactam, antibiotic, aminoglycoside, macrolide, lincosamide, glycopeptide, tetracyline, chloramphenicol, quinolone, fucidin, sulfonamide, trimethoprim, rifamycin, oxaline, streptogramin, lipopeptide, ketolide, polyene, azole, echinocandin, alpha-terpineol, methylisothiazolone, cetylpyridinium chloride, chloroxyleneol, hexachlorophene, chlorhexidine, cationic biguanide, methylene chloride, iodine, iodophore, triclosan, taurinamides, nitrofurantoin, methenamine, aldehydes, azylic acid, rifampycin, silver, benzyl peroxide or silver sulfadiazine. Also useful as antimicrobials are anthracyclines, such as doxorubicin or mitoxantrone, fluoropyrimidines such as 5-fluoroacil, and podophylotoxins, such as etoposide. The salts and the derivatives of all of these are meant to be included as examples of antimicrobial drugs.

In another embodiment, the bioactive is an antifungal drug such as amphotercin B, fluconazole, flucytosine, itraconazole, ketoconazole, nystatin or clotrimazole.

Metals, especially heavy metals, and ionic compounds and salts of these metals, are known to be useful as antimicrobials even in very low amounts or concentrations. These ingredients are said to have an oligodynamic effect, and they are considered oligodynamic. The metals include silver, gold, zinc, copper, cerium, gallium, platinum, palladium, rhodium, iridium, ruthenium, osmium, zinc, bismuth, and others. Other metals with lower atomic weights also have an inhibiting or cidal effect on microorganisms in very low concentrations. These metals include aluminum, calcium, sodium, magnesium, potassium, manganese, and lithium, among others. For present purposes all these metals are oligodynamic metals, and their compounds and ionic ingredients are oligodynamic ingredients. The metals, their compounds and ions, e.g., zinc oxide, silver acetate, silver nitrate, silver chloride, silver iodide and many others, may inhibit the growth of microorganisms, such as bacteria, viruses, or fungi, or they may have cidal effects on microorganisms, such as bacteria, viruses, or fungi, in higher concentrations.

Silver salts are particularly useful for their inhibiting and cidal effects on microorganisms, such as bacteria, viruses and fungi. Such salts include, but are not limited to, silver oxide, silver chloride, silver iodide, silver citrate, silver nitrate, silver lactate, silver acetate, silver propionate, silver salicylate, silver bromide, silver ascorbate, silver laurel sulfate, silver phosphate, silver sulfate, silver benzoate, silver carbonate, silver sulfadiazine, silver gluconate and combinations thereof.

In another embodiment, the bioactive is an analgesic or an anesthetic. Analgesics include naproxen, choline, diflunisal, and salsalate. Other analgesics include non-steroidal antiflammatory agents, such as naproxen, choline, diflunisal, salsalate, fenoprofen, flurbiprofen, ketoprofen, ibuprofen, oxaprozin, diclofenac, indomethacin, sulindac, acetoaminophen, tolmetin, moloxicam, piroxicam, meclofenamate, mefanimic acid, nabumetone, etodelac, keterolac, celecoxib, valdecoxib and rofecoxib, mixtures thereof, and derivatives thereof. Other analgesics include opioids, synthetic drugs with narcotic properties, and narcotics such as alfentanil, buprenorphine, carfentanil, codeine, codeinone, dextropropoxyphene, dihydrocodeine, endorphin, fentanyl, hydrocodone, hydromorphone, methadone, morphine, morphinone, oxycodone, oxymorphone, pethidine, remifantanil, sulfentanil, thebaine, tramadol, and mixtures or derivatives thereof.

Anesthetics which may be used as the bioactive in the device of the present invention include local anesthetics such as paracetamol, bupivacaine, prilocalne, levobupivicaine, dubucaine, ropivacaine, lidocaine, and novocaine.

Of course, the present invention also includes devices including a combination of one or more of the bioactives described above.

### Attachment and Release of the Bioactive

In one embodiment, the devices of the present invention provide for the rapid delivery of the bioactive to the uterine wall. For example, in embodiments where the device contains an uterotonic for the treatment of postpartum hemorrhaging, it is important that the bioactive is released quickly so that hemorrhaging is stopped. In other embodiments, the present devices allow for the controlled release of the hioactive over a prolonged period. For example, where the bioactive is an antimicrobial, controlled release over a prolonged period is preferred.

In various embodiments, at least 90 percent of the bioactive present on or in the device is released into an aqueous physiological environment within 1 minute, 2 minutes, 5 minutes, 10 minutes, 15 minutes, 20 minutes, 30 minutes, 45 minutes, 60 minutes or 90 minutes.

In other embodiments, less than 90 percent of the bioactive present on or in the device is released into an aqueous environment over a period of at least about 6 months, two months, one month, one week, one day, 6 hours, 4 hours, 2 hours, 1 hr, 30 minutes or 15 minutes.

In the embodiment illustrated in FIG. 2, the bioactive is contained in a liquid or gel formulation present in the lumen between inner impermeable balloon 22 and outer balloon 23. The bioactive is delivered through outer balloon 23 to the uterine wall upon inflation of inner balloon 22. In one embodiment, a delivery lumen allows for replenishment of the bioactive when the device is in place within the uterus. Such an embodiment is illustrated in FIG. 3.

In certain embodiments, outer balloon 23 contains pores to allow delivery of the bioactive. In one embodiment, the pores are sized between approximately 0.01 microns and 500 microns. In other embodiments, the pores are sized between 0.1 microns and 100 microns. In yet other embodiments, the pores are sized between 1 micron and 10 microns.

In certain embodiments, a viscous carrier gel is present to help keep the bioactive on the outer surface on balloon 23 as the balloon is inflated and to ensure that the bioactive is effectively delivered to the uterine wall. An absorbent material may also be attached to the outer surface for this purpose.

In certain embodiments, the bioactive is contained in a hydrogel formed from a three-dimensional network of hydrophilic polymer chains that are cross linked through either chemical or physical bonding. In one embodiment, the hydrogel absorbs large amounts of water when placed in the uterus, resulting in rapid release of the bioactive.

Hydrophilic polymers that may be suitable for use are readily and commercially available from, for example, Biosearch Medical Products, Sommerville, N.J.; Hydromer Inc. Branchburg, N.J.; Surmodics, Eden Prairie, Wis.; and STS Biopolymers, Inc., Henrietta, N.Y. For example, the hydrophilic polymer may include, but not be limited to, polyethylene oxide, polyvinyl alcohol, polyvinyl pyrrolidone, polyethylene glycol, carboxylmethyl cellulose, hydroxymethyl cellulose, oxidized dextran, a gelatine, an agarose, a hydrophilic polyurethane, a hydrophilic methacrylate, a hydrophilic methacrylamide or a co-polymer or combination thereof.

In one embodiment, a sleeve containing a mixture of a hydrogel polymer, such as polyvinyl alcohol or polyvinylpyrrolidone, and the bioactive is formed over at least a portion of the balloon surface. The sleeve can also be formed on a mandrel and then placed over the balloon surface. The sleeve can be formed, for example, by extrusion, formed in a mold or by coating a mandrel with a pressure spray system. In certain embodiments, the bioactive is mixed with the polymer and the mixture formed into the sleeve. In certain embodiments, the mixture also included a uv-crosslinking agent, allowing the polymer to be crosslinked using a UV light source. In other embodiments, a material such as polyethylene glycol is added to very the hydration properties of the hydrogel.

The hydrogel may be activated by immersing the covered balloon in an aqueous solution, for example, sterile saline. When placed in a body cavity, the hydrogel swells and stretches with the expanding balloon, releasing the bioactive.

In another embodiment, the bioactive is positioned within the material of the balloon. For example, the bioactive can be mixed with a polymer and extruded to form the balloon. Such a method of manufacture is suitable for those bioactives that are stable under the conditions, particularly the temperature, required for the extrusion process. For example, in one embodiment, a powered base silicone material is mixed with the bioactive in a solvent. The mixture is then extruded at low temperatures with the solvent evaporating as the silicone material cures. Low temperature silicone is utilized so as not to evaporate or inactivate the bioactive.

In another embodiment, a bioactive is imbibed into the material of the balloon. U.S. Pat. No. 5,624,704, which is hereby incorporated by reference, teaches such methods of incorporating a bioactive into the material of a non-metallic device. Briefly, the device is contacted with a solvent containing the bioactive and a penetrating agent. In one embodiment, an alkalinizing agent is added to enhance the reactivity of the material of the device. The solvent is preferably an organic solvent and the penetrating agent is an ingredient that enables the bioactive to permeate the base material of the device and to become deposited within the device.

Examples of suitable organic solvents include, but are not limited to, alcohols (i.e. methanol, ethanol), ketones (acetone, methylethylketone), ethers (tetrahydrofuran), aldehydes (formaldehyde), acetonitrile, acetic acid, methylene chloride and chloroform. The penetrating agent can be any compound that can be used to promote penetration of the bioactive into the material of the device. Examples of suitable compounds are esters (i.e. ethyl acetate, propyl acetate, butyl acetate, amyl acetate, and combinations thereof), ketones (i.e. acetone and methylethylketone), methylene chloride and chloroform. The alkalinizing agent can be an organic and inorganic base including sodium hydroxide, potassium hydroxide, ammonia in water (27% ammonium hydroxide), diethylamine and triethylamine. A high ionic strength salt may act both as an alkalinizing agent and as a penetrating agent. Such salts include sodium chloride, potassium chloride and ammonium acetate.

In another embodiment, the bioactive is applied to the outside surface of the balloon. For example, the bioactive may be applied by spraying, dipping, pouring, pumping, brushing, wiping, vacuum deposition, vapor deposition, plasma deposition, electrostatic deposition, ultrasonic deposition, epitaxial growth, electrochemical deposition or any other method known to those skilled in the art. The bioactive may be applied as a separate layer or may be included in a layer also including a carrier material as described below.

In one embodiment, a bioactive is placed directly on the surface of the balloon and forms the outermost coating layer on the balloon. In another embodiment, the bioactive is coated onto the balloon and one or more barrier layers are placed over at least a portion of the bioactive.

In yet another embodiment, the bioactive is mixed with a carrier material and this mixture applied to the balloon. Alternatively, the carrier material may be applied to the surface of the balloon and the bioactive absorbed into the carrier material. In such configurations, the release of the bioactive may be dependent on factors including the composition, structure and thickness of the carrier material. In one embodiment, the carrier material may contain pre-existing channels, through which the bioactive may diffuse, or channels created by the release of the bioactive, or another soluble substance, from the carrier material.

In other embodiments of the invention, a combination of one or more layers of bioactive, mixtures of carrier material/bioactive, and barrier layers are present on the surface of the balloon. For example, the bioactive may be mixed with a carrier material and coated onto the balloon and then over-coated with one or more barrier layer(s). In yet other embodiments, multiple layers of bioactive, or mixtures of carrier material/bioactive, possibly separated by barrier layers, are present to form a multicoated balloon. In certain embodiments, different bioactives are present in the different layers.

In certain embodiments of the invention, the carrier material and/or the barrier layer comprise a biocompatible polymer. Such polymers include both biostable and biodegradable polymers. Selection of the appropriate polymer for use in the present invention may depend upon the desired rate of release of the bioactive, the porosity of the polymer, and the rate of degradation of the polymer, for example. The coating compositions of the present invention may also include additives, such as diluents, excipients, stabilizers or the like.

In another embodiment, an absorbable mesh is attached to the outside surface of the balloon and the bioactive is absorbed into this mesh. Examples of suitable mesh include mesh cotton cellulose or derivative of cellulose, cotton, cotton derivatives, alginates, dextran and rayon. Such materials may be chosen to absorb body fluids which they come into contact with and in doing so to swell and release the bioactive.

In yet another embodiment, the bioactive is contained within an intermediate space between an outer and an inner balloon. In such an embodiment, the bioactive is preferable dissolved in a liquid or gel. Suitable solvents include water for injection, isotonic saline or other sterile solutions. Polymeric hydrogels may also be used.

The amount of bioactive present on or in the devices of the present invention is such that a therapeutically effective amount of the bloactive is delivered to the patient. In one embodiment, the device contains from about 0.1 μg to about 100 μg of the bioactive per mm² of the gross surface area of the inflated balloon. In another embodiment, the layer of bioactive contains from about 1 μg to about 40 μg of the bioactive 4 per mm² Of the gross surface area of the inflated balloon.

### Methods of Treating a Patient

Another aspect of the present invention provides a method of delivering a drug directly to the uterine wall. Such a method can be of use in the treatment of conditions such as, but not limited to, uterine cancer, endometrial ablation, abnormal uterine bleeding, postpartum hemorrhage and Aschermann's Syndrome.

One embodiment provides for a method of treating postpartum hemorrhaging in a human or veterinary patient. The method combines the application of tamponade pressure on the uterine wall and the application to the uterine wall of a therapeutically effective amount of a bioactive that induces contraction of the uterus (an uterotonic agent).

In one embodiment, the method comprises inserting the balloon portion of the device of the present invention into the uterus, preferably under ultrasound guidance. The balloon is then inflated, preferably with a sterile liquid. In a particularly preferred embodiment, the balloon is inflated with an isotonic saline solution. Gaseous inflation media, such as oxygen nitrogen or carbon dioxide can be used. However, the use of such media involves the danger of gas bubbles entering the blood stream, resulting in serious injury or even death. In addition, liquid media are preferred because such media are not as compressible as gaseous media and allow the balloon to maintain pressure longer.

The balloon is inflated until the outer surface of the balloon contacts and applies pressure to a substantial portion of the uterine wall. In some cases, the application of such pressure can, by itself, provide at least temporary control of bleeding from the wall of the uterus. The balloon is maintained in position for a time sufficient to deliver a therapeutically effective amount of an uterotonic agent from the balloon to the uterine wall. As disclosed above, the uterotonic agent can be present in the material forming the balloon of applied to the outside surface of the balloon. Alternatively, the uterotonic agent may be present in a lumen between an inner and outer balloon, as is illustrated in FIG. 2.

In one embodiment, the balloon is maintained in place for up to 24 hrs. In other embodiments, the balloon is maintained in place for between 1 hr and 24 hrs, between 2 hrs and 12 hrs or between 4 hours and 8 hrs. However, the balloon may be removed at any time if there is excessive bleed or other indications that more aggressive treatment is required.

### EXAMPLE 1

### (Prophetic)—Process for Forming a Sleeve including Polyvinylpyrrolidone for a SOS Bakri Balloon

A deflated Bakri Tamponade balloon (Cook Medical, Inc., Bloomington, Ind.) is coated with a composition including soluble polyvinylpyrrolidone (KOLLIDO® BASF Aktiengesellschaft, Ludwigshafen, Germany) and Oxytocin (SYNTOCINON® available form Sandoz, N.J.) dissolved in a methylene chloride (Sigma-Aldrich Corp. St. Louis, Mo.) solvent. The composition also includes a photo-crosslinker (SurModics, Eden Prarie, Minn.).

The composition is sprayed onto the balloon under a fume hood using a spray gun, such as the Model Number 200 spray gun manufactured by Badger Air-Brush Company, Franklin Park, Ill. After coating, the polymer coating is cured with a UV light source and air dried.

### EXAMPLE 2

### (Prophetic)—Process for Forming a Sleeve including Polyvinylpyrrolidone and Polyethylene glycol (PEG) for a SOS Bakri Balloon

The deflated balloon is spray coated as in Example 1 except that polyethylene glycol is added to the polyvinylpyrrolidone present in the coating solution.

It is to be understood, that the above-described medical devices are merely an illustrative embodiment of the principles of this invention, and that other devices and methods for using them may be devised by those skilled in the art, without departing from the spirit and scope of the invention. It is to be understood that the invention is directed to embodiments both comprising and consisting of the disclosed parts Accordingly, the invention should be limited only by the spirit and scope of the claims.

We claim:

1. A delivery system for a bioactive, the delivery system comprising:
a first inflatable balloon;
a second inflatable balloon positioned within the first inflatable balloon and defining an intermediate space between the first inflatable balloon and the second inflatable balloon;
a catheter having a first lumen, wherein the first lumen is in fluid communication with an interior of the second inflatable balloon; and
a composition comprising a bioactive positioned within the intermediate space, wherein the bioactive is an uterotonic agent.

2. The delivery system of claim 1, wherein the first inflatable balloon is at least partially permeable to the bioactive and the second inflatable balloon is impermeable to the bioactive.

3. The delivery system of claim 1, wherein the bioactive is an agent for the treatment of a condition selected from the group consisting of abnormal uterine bleeding, post-hysteroscopic tamponade, post-partum hemorrhage, Ascherman's syndrome, and endometrial ablation.

4. The delivery system of claim 1, wherein the bioactive is a prostaglandin or a prostaglandin analogue.

5. The delivery system of claim 1, wherein the bioactive is selected from the group consisting of 15-methyl prostaglandin F_{2α}, a synthetic prostaglandin E₁ (PGE₁) analogue, ergometrine maleate, oxytocin and mixtures thereof.

6. The delivery system of claim 1, wherein at least one of the first inflatable balloon and the second inflatable balloon comprises a polymer.

7. The delivery system of claim 6, wherein the polymer is selected from the group consisting of a polyurethane, silicone, natural rubber, synthetic rubber and latex.

8. The delivery system of claim 7, wherein the polymer is a silicone.

9. The delivery system of claim 1, wherein the catheter further comprises a second lumen, wherein the intermediate space is in fluid communication with the second lumen.

10. A delivery system for a bioactive, the delivery system comprising:
a catheter having a lumen;
an inflatable balloon, wherein an inside surface of the inflatable balloon is in fluid communication with the lumen; and
a bioactive associated with the inflatable balloon, wherein the bioactive is an uterotonic agent.

11. The delivery system of claim 10, wherein the uterotonic agent is a prostaglandin or a prostaglandin analogue.

12. The delivery system of claim 10, wherein the uterotonic agent is selected from the group consisting of 15-methyl prostaglandin F_{2α}, a synthetic prostaglandin E₁ (PGE₁) analogue, ergometrine maleate, oxytocin and mixtures thereof.

13. The delivery system of claim 10, wherein the bioactive is positioned within material forming the inflatable balloon.

14. The delivery system of claim 10, wherein the bioactive is positioned on an outside surface of the inflatable balloon.

15. The delivery system of claim 10, further comprising a gauze or resorbable mesh attached to an outside surface of the balloon, wherein the bioactive is within the gauze mesh.

16. The delivery system of claim 10, further comprising a hydrogel sleeve contacting an outside surface of the balloon, wherein the bioactive is contained with the hydrogel sleeve.

17. A method of treating postpartum hemorrhage, the method comprising:
inserting a inflatable balloon into the uterus, wherein the inflatable balloon is attached to a catheter having a lumen in fluid communication with the inflatable balloon and wherein an uterotonic agent is positioned within material forming the inflatable balloon or on an outside surface of the inflatable balloon;
inflating the inflatable balloon to contact and apply pressure to the uterine wall, and
maintaining contact with the uterine wall for a time sufficient to delivery a therapeutically effective amount of the uterotonic agent to the uterine wall.

18. The method of claim 17, wherein the uterotonic agent is a prostaglandin or a prostaglandin analogue.

19. The method of claim 17, wherein the uterotonic agent is selected from the group consisting of 15-methyl prostaglandin F_{2α}, a synthetic prostaglandin E₁ (PGE₁) analogue, ergometrine maleate, oxytocin and mixtures thereof.

20. The method of claim 17, wherein a therapeutically effective amount of the uterotonic agent is delivered to the uterine wall within 60 minutes.
